(19) United States
(12) Patent Application Publication
(10) Pub. No.: US 2022/0267810 A1
(43) Pub. Date: Aug. 25, 2022

(54) Method for reduction of the carbon intensity of a fermentation process

(71) Applicant: Carbon Sink LLC, Arlington, VA (US)

(72) Inventors: Maxim Lyubovsky, Falls Church, VA (US); Randel E. Roy, Severna Park, MD (US); Stephen Robert Meyer, McLean, VA (US); James Patrick McVaney, Arlington, VA (US); Henry P. Aszklar, Jekyll Island, GA (US)

(73) Assignee: Carbon Sink LLC, Arlington, VA (US)

(21) Appl. No.: 17/675,324

(22) Filed: Feb. 18, 2022

Related U.S. Application Data
(60) Provisional application No. 63/151,857, filed on Feb. 22, 2021.

(51) Int. Cl. C12P 7/06; C25B 1/04; C25B 9/67; C25B 15/08; C07C 1/12; C07C 31/04

(52) U.S. Cl. CPC C12P 7/06; C25B 1/04; C25B 9/67; C25B 15/081; C07C 1/12; C07C 31/04

(57) ABSTRACT

The invention relates to a method for reduction of the carbon intensity of an ethanol production process by utilizing waste heat from a co-located water electrolysis system for heating duty of the ethanol plant; using oxygen (O₂) produced by the water electrolysis system for oxycombustion of hydrocarbon fuel to produce the required thermal energy; and capturing carbon dioxide (CO₂) from the fermentation process and from the oxycombustion process, combining it with hydrogen (H₂) produced by electrolysis, to produce additional hydrocarbon fuels and durable chemicals.

### CROSS REFERENCE TO RELATED APPLICATIONS

Not applicable.

### STATEMENT OF FEDERALLY SPONSORED RESEARCH

Not applicable.

### NAMES OF PARTIES TO A JOINT RESEARCH AGREEMENT

Not applicable.

### REFERENCE TO A SEQUENCE LISTING, A TABLE, OR A COMPUTER PROGRAM

Not applicable.

### BACKGROUND OF THE INVENTION

Lowering carbon intensity of the fuels used in transportation is an important goal facing society. One of the avenues for production of transportation fuels with low carbon intensity is through production of biofuels through the fermentation of biomass. One of the most common industrial fermentation processes is ethanol production. Ethanol can be produced from a variety of biomass sources, such as sugar cane, grain-based feedstocks, such as corn, or cellulosic feedstocks, such as switchgrass.

Much of the carbon in biofuels originates from CO₂ captured directly from air in the process of biomass growth, so that net zero carbon emission is achieved when the fuel is combusted in an internal combustion engine. Negative emissions are achieved when the biofuel is converted into durable chemical materials. Currently, there are significant emissions of carbon dioxide from an ethanol plant, which originate from two distinct sources. First, there is CO₂ formed in the fermentation process itself. For example, the process of fermentation of sucrose into ethanol can be represented as C₆H₁₂O₆→2 C₂H₅OH+2 CO₂. Therefore, about one third of the carbon accumulated in the biomass is converted back into CO₂, which is usually vented from the fermentation reactors into the atmosphere. The second source of CO₂ emissions from an ethanol plant is from the combustion of fuel, in which, natural gas (“NG”) is most commonly used. The heat produced by this combustion is used in several places of the ethanol production process, such as the cooker for liquefaction of starch-containing slurry, the distillation column, the ring dryer for drying of wet solids, etc.

This invention provides a method for the reduction of the carbon intensity of an ethanol production plant through the reduction in carbon dioxide from both sources of the ethanol plant.

### FIELD OF THE INVENTION

The present invention relates to a method for the production of ethanol wherein there is a reduction of the carbon intensity of an ethanol production process by utilizing waste heat from a co-located water electrolysis system.

### BRIEF SUMMARY OF THE INVENTION

The present invention relates to a method for reduction of the carbon intensity of an ethanol production process by utilizing waste heat from a co-located water electrolysis system for heating duty of the ethanol plant; using oxygen (O2) produced by the water electrolysis system for oxycombustion of hydrocarbon fuel to produce the required thermal energy; and capturing carbon dioxide (CO₂) from the fermentation process and from the oxycombustion process, combining it with hydrogen (H₂) produced by electrolysis, to produce additional hydrocarbon fuels and durable chemicals.

### DESCRIPTION OF RELATED ART

There has been work in this area because of the decreasing cost of renewable electricity. Some references of interest include:

Lyubovsky, M. “Production of fuel from air to establish sustainable carbon cycle for zero-emissions economy.” DOE Presentation. January 2019.

Lyubovsky, M. Shifting the Paradigm: synthetic liquid fuels offer vehicle for monetizing wind and solar energy. J. Energy Security. 2017.

### BRIEF DESCRIPTION OF THE DRAWING

FIG. 1 shows the first embodiment of the invention. FIG. 1 shows a number of streams and process units.

10—Water electrolysis system

11—Low-carbon electricity supply

12—Water fed to the electrolysis system

13—Electrolysis system waste heat

14—Hydrogen output

15—Oxygen output

20—Ethanol plant

21—Carbohydrate feed to ethanol plant

26—Ethanol plant product output

FIG. 2 shows a schematic representation of the second embodiment of the invention that shows a number of process units and streams.

110—Water electrolysis system

111—Low-carbon electricity supply

112—Water feed to electrolysis

113—Electrolysis system waste heat

114—Hydrogen output

115—Oxygen output

120—Ethanol plant

121—Carbohydrate feed to ethanol plant

125—CO₂ captured from ethanol plant

126—Ethanol plant product output

130—Reactor system for combining CO₂ and H₂

131—Synthetic hydrocarbon product output

FIG. 3 shows a schematic representation of the third embodiment of the invention with a number of process units and streams.

210—Water electrolysis system

211—Low-carbon electricity supply

212—Water feed to electrolysis

213—Electrolysis system waste heat

214—Hydrogen output

215—Oxygen output

220—Fermentation plant

221—Carbohydrate feed to ethanol plant

222—Ethanol plant oxycombustion burner

223—Ethanol plant heating fuel supply

225—CO₂ captured from ethanol plant

226—Ethanol plant product output

230—Reactor system combining CO₂ and H₂

231—Synthetic hydrocarbon product output

### DETAILED DESCRIPTION OF THE INVENTION

The method of this invention provides for reduction of carbon intensity of an ethanol plant by: 1) substituting heat produced by combustion of hydrocarbon fuels with the waste heat produced by a water electrolysis system; 2) utilizing oxygen produced by the water electrolysis system for oxycombustion of the hydrocarbon fuel to produce additional heat; and 3) capturing CO₂ emitted by the fermentation process and the oxycombustion process and combining it with the hydrogen produced by water electrolysis in a hydrocarbon synthesis reactor to produce a renewable liquid hydrocarbon product.

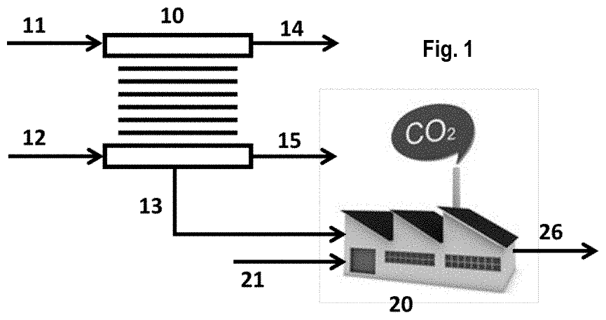

Referring to FIG. 1 of the first embodiment of the method of this invention comprises the following. Locating at least one electrolysis system 10 which utilizes water 12 and a low-carbon electric power source 11 to produce hydrogen 14 and oxygen 15 in close proximity of the ethanol plant 20, which converts the carbohydrate feed stream 21 into ethanol 26; placing the water electrolysis systems 10 in close proximity to the ethanol plant 20; and utilizing at least a part of the waste heat 113 from the water electrolysis systems 10 to provide at least part of the heating duty of the ethanol plant 20. The term “locating” means to build, construct, or otherwise situate in a particular place. The term “close proximity” means within a short distance where it is feasible to send streams back and forth between the ethanol plant and the electrolysis system. A distance of less than one mile is considered close proximity.

Low-carbon electric power 11 can be supplied by (1) the electricity grid in the region if it has a high penetration of renewable or nuclear based energy supplying it or (2) by directly connecting to renewable energy sources. The carbon intensity of the low-carbon electric supply will be lower than the carbon intensity of natural gas (NG) combustion, commonly used for providing heat for the ethanol plant, which is about 200 kg CO₂/MWh. Preferably, the low-carbon electric energy 11 is supplied by directly connecting the water electrolysis system 10 to a carbon free renewable power system, such as wind farm, solar panels or other known types.

The water electrolysis systems 10 can be of any known type. Three water electrolysis technologies are currently available—alkaline electrolysis, proton exchange membrane electrolysis (PEM) and Solid Oxide electrolysis (SOEC). The system properties and operating conditions for these water electrolysis technologies are well known in the art and are described in multiple publications. The particular water electrolysis technology selected for integration with a specific application will be determined, in part, based on the temperature requirements of the ethanol plant 20 and the intermittency of the low-carbon electric energy supply 11. Alkaline electrolysis is the most mature technology. It operates in the temperature range between 60 to 90° C. and can be started and stopped rapidly in response to variations in the intermittent supply of renewable energy. PEM electrolysis systems in the megawatt power range are commercially available from several manufacturers. PEM electrolysis operates in the temperature range between 60 to 80° C. PEM systems can also be started and stopped rapidly and, therefore, can be integrated with intermittent renewable power systems. SOEC electrolysis systems operate at much higher temperatures, between 600 to 900° C., have higher energy efficiency per unit of hydrogen production and can provide waste heat of higher quality than other electrolysis technologies. SOFC system, though, are not as mature as PEM and alkaline technologies, and require much longer times for starting and stopping. Considering the last point, they are not well suited to the intermittency of renewable energy. Thus, they should only be used with continuous low-carbon electric energy supplied by a low-carbon based electricity grid.

As is well known, water electrolysis systems split water to produce hydrogen and oxygen in 2:1 molar ratio of hydrogen to oxygen. The amount of hydrogen and oxygen produced is proportional to the amount of electrical energy supplied to the electrolysis system. The correlation between the supplied electrical energy and the amount of hydrogen and oxygen produced is specific to the electrolysis system supplier. The electrolysis system also produces waste heat. Depending on the type of the water electrolysis system, the amount of the waste heat will be between 20% and 40% of the electrical energy supplied to the electrolysis system. The waste heat can be removed from the water electrolysis system by any heat carrying fluid known in the art. For alkaline or PEM electrolysis systems hot water is commonly used as the heat removing fluid. For SOFC electrolysis systems, steam or oil are often employed.

The ethanol plant 20, can be any type of a known fermentation process, which utilizes carbohydrate feed to produce a fermentation product with CO₂ as a by-product, and requires process heat input at different stages of the process. Wet mill and dry mill ethanol plants are the most common example of commercial ethanol plants in the USA.

In the method of the first embodiment of this invention the water electrolysis system 10 should be placed in reasonably close proximity to the ethanol plant 20, so that at least the portion of the heat carrying fluid from the water electrolysis system 10 can flow to the ethanol plant 20, providing at least part of the required heating duty for the ethanol plant, which would otherwise normally be supplied by combustion of a hydrocarbon fuel, such as NG. Use of this heat carrying fluid from the electrolysis system reduces the CO₂ emissions which are produced in the combustion process. As anyone skilled in the art would recognize, the heat carrying fluid of any type cannot be transported over long distances without losing substantial amount of the heat. Therefore, the water electrolysis system 10 and the ethanol plant 20 of this invention should be located in close proximity to each other.

The low-carbon electric energy 11 comprising a large fraction of renewable energy will possibly be intermittent in nature. Thus, it may not be available continuously. Waste heat from the water electrolysis system 10 will also be available only when low-carbon electric power supply 11 is available. As people familiar with operation of the ethanol plants would recognize, some operations in the ethanol plant can be deferred until the heat supply from the low-carbon electric power supply 11 and the waste heat from the water electrolysis system 10 are available. A by-product of the fermentation process is wet distiller's grain. This is most often dried to allow for long term storage without spoiling. Drying the wet distillers' grain consumes a large fraction of the heat required by the ethanol plant. Wet distillers' grain can be accumulated and then dried when the low-carbon electric power supply is available.

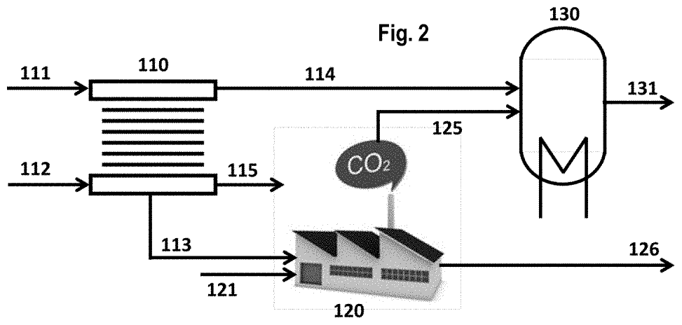

The second embodiment of the method of this invention refers to FIG. 2. Similar components in the figures have similar numbers. The method of the second embodiment of this invention comprises locating at least one electrolysis system 110 which utilizes water feed 112 and a low-carbon electric power source 111 to produce hydrogen 114 and oxygen 115 in close proximity to the ethanol plant 120, which converts carbohydrate feed stream 121 into the ethanol plant product output 126; placing the water electrolysis systems 110 in reasonably close proximity to the ethanol plant 120; utilizing at least a portion of the waste heat 113 from the water electrolysis systems 110 for the heating duty of the ethanol plant 120; and capturing the CO₂ emitted by the ethanol plant 125 and combining it with the hydrogen produced by the water electrolysis system 114 in a hydrocarbon synthesis system 130 to produce liquid hydrocarbon product 131.

The fermentation process emits a nearly pure stream of CO₂ which contains only water vapor and small amounts of impurities, such as hydrogen sulfide and silicates. These impurities can be removed from the CO₂ captured from the fermentation process 125 by any know gas purification technology, such as absorbent beds, membranes, PSA or others.

Purified CO₂ captured from the fermentation process 125 and hydrogen from the water electrolysis system 114 are further supplied to the reactor system 130 which combines CO₂ and H2 to produce a hydrocarbon product 131. The hydrocarbon product 131 can be any chemical compound having general formula CₓH_{y}O_{z}. These can be different classes of chemical compounds, such as for example, alkanes, alkenes, aromatics, alcohols, aldehydes, and others, or mixtures of different compounds. Through known petrochemical processes the hydrocarbon product 131 can be further upgraded to conventional fuels and durable material for long term carbon sequestration. The reactor system 130 may be any of the many types processes known in the art for combining CO₂ with hydrogen, such as a methanation reactor, a reverse water gas shift reactor followed by Fischer-Tropsch synthesis, a bio-chemical reactor, or any other of the known type. The reactor system based on a methanol synthesis reactor, producing methanol as the hydrocarbon product 131, is the preferred type of the system for this invention.

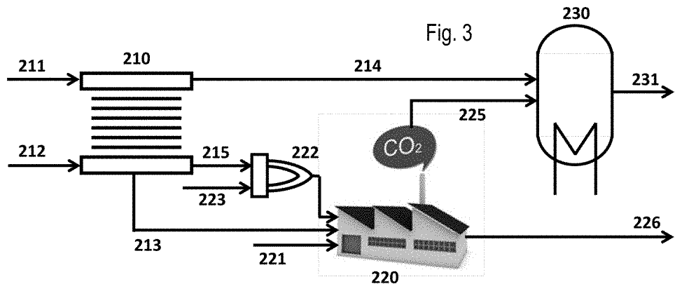

The third embodiment of the method of this invention refers to the FIG. 3. Similar components in the figures have similar numbers. The method of the third embodiment of this invention comprises locating at least one electrolysis system 210 which utilizes water feed 212 and low-carbon electric power source 211 to produce hydrogen 214 and oxygen 215 in close proximity to the ethanol plant 220, which converts carbohydrate feed stream 221 into the ethanol plant output 226; placing the water electrolysis systems 210 in close proximity to the ethanol plant 220; utilizing at least a portion of the waste heat 213 from the water electrolysis systems 210 for the heating duty of the ethanol plant 220; utilizing at least a portion of the oxygen 215 for oxycombustion of the fuel supply 223 used in the ethanol plant, and capturing the CO2 emitted by the fermentation process 125 and the oxycombustion burner 222, and combining the captured CO₂ with the hydrogen produced by the water electrolysis system 214 in a hydrocarbon synthesis system 230 to produce a liquid hydrocarbon product 231.

Oxycombustion is a well know method of generating heat where hydrocarbon fuel is combusted using pure oxygen as an oxidizer, so that only CO₂ and steam are produced in the combustion process, as for example, shown by the equation for oxycombustion of methane.

$${CH}_{4} + 2 {O}_{2} = {CO}_{2} + 2 {H}_{2} O$$

Liquid water can be easily condensed from the exhaust of oxycombustion leaving only CO₂ and possibly minor impurities originating from the impurities that might be present in the fuel. This CO₂ can be captured and combined with the CO₂ captured from the CO₂ emitted by the fermentation process in the feed to the hydrocarbon synthesis system 230 where it is combined with hydrogen produced by the water electrolysis system 214 to produce a liquid hydrocarbon product 231.

Carbon Intensity can be calculated by a number of methods. Argonne National Labs has developed the GREET Model to calculate carbon intensities of fuels produced by various processes. The California Air Resources Board has developed a specific version of the GREET model and all low carbon fuels sold in the state receive a Carbon Intensity score from that model. Specifically, the California GREET model referred to in this application is the CA_GREET 3.0 model that was adopted by the Air Resources Board in September 2018. The current approved ethanol fuel pathways from corn or corn kernels via fermentation have a range of carbon intensities from 53 to 85. Ethanol produced by the processes described here have carbon intensities preferably lower than 50, more preferably less than 40, and even more preferably less than 30.

### Example 1

### Example of the First Embodiment of the Invention.

In an example of the first embodiment, an ethanol plant having 50 million gallons per year ethanol production, which is a common size of a commercial ethanol plant in the USA, is used as the basis. The values of heat and CO₂ emissions in this example are shown in Table 1 and are based on the results of the system modelled using a VMGSim thermodynamic modeling software.

A modern ethanol plan uses about 35,000 BTUs per gallon of ethanol produced in various stages of the production process, such as corn meal cooking, which breaks starch into sugars; ethanol separation in the distillation column; drying wet cake into distillers' dry grains and solubles (DDGS) for livestock feed; etc. Assuming that NG is used as the fuel for heating, 117 lb of CO₂ is produced per each MMBTU of heat which results in total CO₂ emissions of 92,857 tonnes per year from the ethanol plant thermal energy requirements under normal operation. Together with 150,000 tonnes/yr of CO₂ emitted from the fermentation process, this results in the total emissions from an ethanol plant of 242,857 tonnes CO₂ per year (Table 1).

Under the first embodiment of this invention, 1.9 TWh of renewable electric energy is required for the electrolyzers to produce the required 34,549 tonnes of hydrogen. This equates to 220 MW of electrical power supply in continuous operation. At 73% efficiency, the electrolyzers produce 513,270 MWh (of waste heat, which when supplied to the ethanol plant may replace 1,750,000 MMBTU of the heat produced by burning NG. This waste heat utilization, therefore, eliminates the release of 92,857 tonnes of CO₂ per year, or about 4.1 lb of CO₂ per gallon of ethanol produced.

TABLE 1
Ethanol plant CI reduction by the first embodiment of the invention

| | | |
|---|---|---|
| Ethanol plant production rate | 50 | MM gal/yr |
| CO₂ emissions from fermentation process | 150000 | tonne/yr |
| Thermal energy used in corn ethanol plant | 35000 | BTU/gal |
| Thermal energy requirement per year | 1750000 | MMBTU/yr |
| | 513270 | MWh/yr |
| CO₂ emissions from NG combustion | 117 | lb/MMBTU |
| CO₂ emissions from the ethanol plant heating | 205 | MM lb/yr |
| | 92857 | tonne/yr |
| Total CO₂ emissions from the ethanol plant | 242857 | tonne/yr |
| | 10.7 | lb/gal |
| Renewable energy to electrolyzer | 1911209 | MWh/yr |
| H2 production (73% efficiency) | 34549 | tonne/yr |
| O2 production | 271428 | tonne/yr |
| Waste heat to the ethanol plant | 513270 | MWh/yr |
| CO2 emissions reduction | 92857 | tonne/yr |
| | 4.1 | lb_CO₂/gal |
| Remaining CO2 emissions | 150000 | tonne/yr |
| | 6.6 | lb_CO₂gal |

This process results in a significant Carbon Intensity reduction for the ethanol product as determined by the CA-GREET model.

### EXAMPLE 2

### Example of the Second Embodiment of the Invention.

This example refers to the same ethanol plant having 50MM gallons per year production rate as in the first example. Under the second embodiment of this invention, 1.1 TWh of renewable electric energy is required for the electrolyzers to produce the required 20,000 tonnes of hydrogen. This equates to 125 MW of power supply in continuous operation. The electrolyzers also produce 290,938 MWh of waste heat, which when supplied to the ethanol plant, replaces 991,000 MMBTU of the heat produced by burning NG. This waste heat utilization eliminates the release of 52,643 tonnes of CO₂ per year.

150,000 tonnes/yr of CO₂ emitted from the fermentation process is captured and combined with hydrogen produced by the electrolyzers to produce 103,000 tonnes/yr of methanol. Total reduction in CO₂ emissions from the ethanol plant is then 202,634 tonnes CO₂ per year, or 8.9 lb of CO₂ per gallon of ethanol produced.

TABLE 2
Ethanol plant CI reduction by the second embodiment of the invention

| | | |
|---|---|---|
| Ethanol plant production rate | 50 | MM gal/yr |
| CO₂ emissions from fermentation process | 150000 | tonne/yr |
| Thermal energy used in corn ethanol plant | 35000 | BTU/gal |
| Thermal energy requirement per year | 1750000 | MMBTU/yr |
| | 513270 | MWh/yr |
| CO₂ emissions from NG combustion | 117 | lb/MMBTU |
| CO₂ emissions from the ethanol plant heating | 205 | MM lb/yr |
| | 92857 | tonne/yr |
| Total CO₂ emissions from the ethanol plant | 242857 | tonne/yr |
| | 10.7 | lb/gal |
| Renewable energy to electrolyzer | 1083333 | MWh/yr |
| H₂ production | 19583 | tonne/yr |
| O₂ production | 153854 | tonne/yr |
| Methanol produced from H₂ and CO₂ | 103125 | tonne/yr |
| Waste heat from electrolyzer to ethanol plant | 290938 | MWh/yr |
| CO₂ reduction from NG replacement | 52634 | tonne/yr |
| Total CO₂ reduction | 202634 | tonne/yr |
| | 8.9 | lb_CO₂/gal |
| Remaining CO₂ emissions | 40223 | tonne/yr |
| | 1.8 | lb_CO₂/gal |

This process results in ethanol with a significantly reduced Carbon Intensity as determined by the CA-GREET model.

### EXAMPLE 3

### Example of the Third Embodiment of the Invention.

This example refers to the same ethanol plant having 50MM gallons per year production rate as in the first and second examples.

Under the third embodiment of this invention, 1.3 TWh of renewable electric energy, is required to produce the required 23,500 tonnes of hydrogen. This equates to 150 MW of power supply in continuous operation. The electrolyzers also produce 348,706 MWh of waste heat, which when supplied to the ethanol plant replaces 1,190,000 MMBTU of the heat produced by burning NG. This waste heat utilization eliminates the release of 53,000 tonnes CO₂ per year.

In addition, 43,280 tonnes/yr of oxygen which is produced by the water electrolysis is used for oxycombustion of NG to produce 560,000 MMBTU/yr of process heat, eliminating 30,000 tonne/yr of CO₂ from the heating process.

Both 150,000 tonne/yr of CO₂ emitted from the ethanol fermentation process and 30,000 tonne/yr of CO₂ from the heating process are captured and combined with the hydrogen produced by the electrolyzers to produce 124,000 tonne/yr of methanol. In this example, all of the CO₂ emissions from the ethanol plant are eliminated with total reduction in CO₂ emissions of 242,857 tonne CO₂ per year, or 10.7 lb of CO₂ per gallon of ethanol produced.

TABLE 3
Ethanol plant CI reduction by the third embodiment of the invention

| | | |
|---|---|---|
| Ethanol plant production rate | 50 | MM gal/yr |
| CO2 emissions from fermentation process | 150000 | tonne/yr |
| Thermal energy used in corn ethanol plant | 35000 | BTU/gal |
| Thermal energy requirement per year | 1750000 | MMBTU/yr |
| | 513270 | MWh/yr |
| CO₂ emissions from NG combustion | 117 | lb/MMBTU |
| CO₂ emissions from the ethanol plant heating | 205 | MM lb/yr |
| | 92857 | tonne/yr |
| Total CO₂ emissions from the ethanol plant | 242857 | tonne/yr |
| | 10.7 | lb/gal |
| Renewable energy to electrolyzer | 1298440 | MWh/yr |
| H₂ production | 23472 | tonne/yr |
| O₂ production | 184403 | tonne/yr |
| CO₂ used in methanol synthesis | 179784 | tonne/yr |
| Methanol produced from H₂ and CO₂ | 123602 | tonne/yr |
| Waste heat from electrolyzer to ethanol plant | 348706 | MWh/yr |
| CO₂ reduction from NG replacement | 63085 | tonne/yr |
| Heat from NG oxycombustion | 164564 | MWh/yr |
| O₂ for oxycombustion @0.263 tonne/MWh | 43280 | tonne/yr |
| CO₂ emitted from NG oxycombustion | 29786 | tonne/yr |
| Total CO₂ reduction | 242869 | tonne/yr |
| | 10.7 | lb_CO₂/gal |
| Remaining CO₂ emissions | 0 | tonne/yr |
| | 0 | lb_CO₂/gal |

This process results in ethanol with a significantly reduced Carbon Intensity as determined by the CA-GREET model. As can be seen in the examples, the total reduction of CO₂/gallon of ethanol is between 4 and 15 lb CO₂/gallon

What is claimed is:

1. A method for reducing carbon intensity of a fermentation process comprising:
a. Locating at least one water electrolysis system which utilizes low-carbon electric power supply to produce hydrogen and oxygen in close proximity to the ethanol plant;
b. Placing the water electrolysis systems in close proximity to the ethanol plant, and
c. Using at least part of the waste heat from the water electrolysis systems for heating duty of the ethanol plant.

2. A method of claim 1 where the heating duties of the fermentation process are deferred to accommodate for the intermittent nature of the low-carbon electric power supply.

3. A method of claim 2 where the fermentation process is an ethanol plant.

4. A method of claim 1 where the CO₂ emitted by the fermentation process is captured and combined with the hydrogen produced by the water electrolysis system in a hydrocarbon synthesis system to produce liquid hydrocarbon product.

5. A method of claim 4 where the hydrocarbon synthesis system produces methanol.

6. A method of claim 4 where at least part of the oxygen output produced by the water electrolysis systems is used for oxycombustion of hydrocarbon fuel to provide at least a portion of the heating duty for the ethanol plant. CO₂ emitted by the oxycombustion process is captured and combined with the CO₂ emitted by the fermentation process and with the hydrogen produced by the water electrolysis system in a hydrocarbon synthesis system to produce liquid hydrocarbon product.

7. A low carbon intensity fuel comprising ethanol wherein the carbon intensity is an amount between 0 g CO₂eq/MJ and 50 g CO₂eq/MJ as determined by the CA-GREET model.

8. The low carbon intensity fuel of claim 7 where the carbon intensity is an amount between 0 g CO₂eq/MJ and 40 g CO₂eq/MJ.

9. The low carbon intensity fuel of claim 7 where the carbon intensity is an amount between 0 g CO₂eq/MJ and 30 g CO₂eq/MJ.

10. The process for the production of low carbon intensity fuel of claim 7 where the ethanol is produced by the process comprising:
a. Locating at least one water electrolysis system which utilizes low-carbon electric power supply to produce hydrogen and oxygen in close proximity to the ethanol plant;
b. And, utilizing at least a fraction of the waste heat from the water electrolysis systems for heating duty of the ethanol plant;
c. Wherein the reduction in CO₂ in the carbon intensity of the ethanol is between 4 and 15 lb CO₂/gallon of ethanol.

11. The process for the production of ethanol comprising:
a. Water is electrolyzed to produce a first stream comprising hydrogen wherein the electrolyzer is powered by renewable electricity;
b. A second stream comprising carbon dioxide is received from an ethanol fermentation process;
c. The first stream comprising hydrogen and the second stream comprising carbon dioxide are reacted to produce a stream comprising methanol;
d. Wherein waste heat from the water electrolysis system is used as heating duty in the ethanol plant.
